(19) United States
(12) Patent Application Publication
(10) Pub. No.: US 2005/0026273 A1
(43) Pub. Date: Feb. 3, 2005

(54) Reactor with memory component

(76) Inventors: Andrey J. Zarur, Winchester, MA (US); Ian K. MacGregor, Merrimack, NH (US); Todd A. Basque, Danvers, MA (US); Seth T. Rodgers, Somerville, MA (US); A. Peter Russo, Woburn, MA (US); Sean J. Leblanc, Westminster, MA (US)

Correspondence Address: Timothy J. Oyer, Ph.D.;Wolf, Greenfield & Sacks, P.C., 600 Atlantic Avenue, Boston, MA, 02210, US

(21) Appl. No.: 10/863,636

(22) Filed: Jun. 7, 2004

Related U.S. Application Data
(63) Continuation-in-part of application No. 10/457,017, filed on Jun. 5, 2003, now abandoned.
(60) Provisional application No. 60/499,124, filed on Aug. 29, 2003.

(51) Int. Cl. C12M 1/36

(52) U.S. Cl. 435/286.1; 422/63; 435/305.3; 435/288.5

(57) ABSTRACT

The present invention provides techniques for conveniently and reliably storing and/or retrieving data associated with a chemical, biological, or biochemical chip, reactor, or reaction system. The data can pertain to the reactor; to chemical, biological, or biochemical species introduced into, taken from, or otherwise associated with the reactor; to conditions to which the reactor and/or some or all of its contents has been, is being, or will be exposed to, or the like. Various aspects of the present invention relate to memory and data storage components suitable for use in chips or other reaction systems. These components may include silicon integrated circuits, magnetic media, optical media, radio-frequency tags, smart cards, bar-codes and other kinds of data storage devices. The chip may contain a reaction site having a volume of less than about 2 ml. In some embodiments, the chip may be constructed in such a way as to be able to support a living cell. The chip may be used for imaging or analysis, or the chip may be used to facilitate a chemical or biological reaction, which may be light-sensitive or light-activated in certain cases. Other facilitated reactions may include the production and/or consumption of a chemical or biological species. In some embodiments, the chip may include more than one component or component type, and/or more than one reaction site.

### RELATED APPLICATIONS

This application is a continuation-in-part of co-pending U.S. patent application Ser. No. 10/457,017, filed Jun. 5, 2003, entitled “System and Method for Process Automation,” by Seth T. Rodgers, et al. This application also claims priority to U.S. Patent Application Ser. No. 60/499,124, filed Aug. 29, 2003, entitled “Reactor with Memory Component,” by Andrey J. Zarur, et al. Each of these applications is incorporated herein by reference.

### FIELD OF THE INVENTION

This invention relates to memory and data storage components and, in particular, to memory and data storage components for use in reactors, chips, and/or reaction systems.

### BACKGROUND OF THE INVENTION

A wide variety of reaction systems are known for the production of products of chemical and/or biochemical reactions. Chemical plants involving catalysis, biochemical fermenters, pharmaceutical production plants, and a host of other systems are well known. Biochemical processing may involve the use of a live microorganism (e.g., cells) to produce a substance of interest.

Cells are cultured for a variety of reasons. Increasingly, cells are cultured for proteins or other valuable materials they produce. Many cells require specific conditions, such as a controlled environment. The presence of nutrients, metabolic gases such as oxygen and/or carbon dioxide, humidity, as well as other factors such as temperature, may affect cell growth. Cells require time to grow, during which favorable conditions must be maintained. In some cases, such as with particular bacterial cells, a successful cell culture may be performed in as little as 24 hours. In other cases, such as with particular mammalian cells, a successful culture may require about 30 days or more.

Typically, cell cultures are performed in media suitable for cell growth and containing necessary nutrients. The cells are generally cultured in a location, such as an incubator, where the environmental conditions can be controlled. Incubators traditionally range in size from small incubators (e.g., about 1 cubic foot) for a few cultures up to an entire room or rooms where the desired environmental conditions can be carefully maintained.

Recently, as described in International Patent Application Ser. No. PCT/US01/07679, published on Sep. 20, 2001 as WO 01/68257, entitled “Microreactors,” incorporated herein by reference, cells have also been cultured on a very small scale (i.e., on the order of a few milliliters or less), so that, among other things, many cultures can be performed in parallel.

### SUMMARY OF THE INVENTION

This invention generally relates to memory and data storage components for use in reactors, chips, and/or reaction systems. A variety of reactors, chips, and reaction systems are provided, as well as methods involving the storage of data in components of reactors, chips, and reaction systems. The subject matter of this invention involves, in some cases, interrelated products and/or uses, alternative solutions to a particular problem, and/or a plurality of different uses of a single system or article.

In one aspect, the invention includes an apparatus. In one set of embodiments, the apparatus includes a chemical, biological, or biochemical chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component. The apparatus, in another set of embodiments, includes a chip comprising a predetermined reaction site, and at least one component where data can be stored in a volatile or a non-volatile form, and, in some cases, from where data can be retrieved. In yet another set of embodiments, the apparatus includes a chip comprising a predetermined reaction site, and at least one component to which data pertaining to the reaction site can be stored and/or from data can be retrieved. The apparatus, in yet another set of embodiments, includes a chip comprising a predetermined reaction site that has a volume of less than about 2 ml or less than about 1 ml l, where the chip has at least one substantially cytophilic or cytophobic surface, and where the apparatus also includes at least one memory and/or data storage component where data related to or associated with the reaction site can be stored to and/or retrieved from.

In one set of embodiments, the apparatus includes a chemical, biological, or biochemical chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component is constructed of a magnetic medium. In another set of embodiments, the apparatus includes a chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component includes a writeable or re-writeable compact disc (CD-R or CD-RW) or digital versatile disc (DVD+R, DVD−R, DVD+RW or DVD−RW), or a component similar to a compact disc or a digital versatile disc. In yet another set of embodiments, the apparatus includes a chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component includes flash memory. In still another set of embodiments, the apparatus includes a chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component includes a silicon-based or semiconductor-based integrated circuit. In another set of embodiments, the apparatus includes a chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component includes a radio-frequency tag or other wireless device.

In another set of embodiments, the apparatus includes a chemical, biological, or biochemical chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component includes a magnetic strip. In still another set of embodiments, the apparatus includes a chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component includes a barcode system. In another set of embodiments, the apparatus includes a chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component includes a smart media card. In yet another set of embodiments, the apparatus includes a chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component includes a memory stick.

In one set of embodiments, the apparatus includes a chemical, biological, or biochemical chip comprising a predetermined reaction site having a volume of less than about 2 ml or less than about 1 ml, where the predetermined reaction site is constructed and arranged to maintain at least one living cell at the site, and where the apparatus also includes at least one memory and/or storage component, where the component is capable of undergoing heat and/or moisture treatment (for example, in an autoclave or an incubator) without losing its data storage capabilities. In another set of embodiments, the apparatus includes a chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component is capable of undergoing gamma-ray treatment (for example, in a gamma-ray sterilization chamber) without losing its data storage capabilities. In yet another set of embodiments, the apparatus includes a chip comprising a predetermined reaction site having a volume of less than about 2 ml or less than about 1 ml, where the predetermined reaction site is constructed and arranged to maintain at least one living cell at the site, and where the apparatus also includes at least one memory and/or data storage component, where the component is capable of undergoing ultraviolet light treatment (for example, in a UV sterilizer) without losing its data storage capabilities.

The invention also includes a system in another aspect. In one set of embodiments, the system is defined, at least in part, by a chemical, biological, or biochemical chip comprising a predetermined reaction site and a memory and/or data storage component, and an external reader capable of retrieving data from the component. The system includes, in another set of embodiments, a chip comprising a predetermined reaction site and a memory and/or data storage component, and an external writer capable of writing data to the component. In still another set of embodiments, the system includes a chip comprising a predetermined reaction site and a memory and/or data storage component, and an external reader/writer capable of writing data to and/or retrieving data from the component.

In another set of embodiments, the system includes a chemical, biological, or biochemical chip comprising a predetermined reaction site having a volume of less than about 2 ml or less than about 1 ml and a memory and/or data storage component capable of storing data relating to and/or associated with the reaction site, where the predetermined reaction site is constructed and arranged to maintain at least one living cell at the site, and where the system also includes an external reader/writer capable of writing data to and/or retrieving data from the component.

In one set of embodiments, the system includes a chemical, biological, or biochemical chip comprising a predetermined reaction site having a volume of less than about 2 ml or less than about 1 ml and a memory and/or data storage component capable of storing data, where the predetermined reaction site is constructed and arranged to maintain at least one living cell at the site, and where the system also includes an external reader/writer capable of writing data to and/or retrieving data from the component by establishing an electrical contact with the component. In another set of embodiments, the system includes a chip comprising a predetermined reaction site having a volume of less than about 2 ml or less than about 1 ml and a memory and/or data storage component capable of storing data, where the predetermined reaction site is constructed and arranged to maintain at least one living cell at the site, and where the system also includes an external reader/writer capable of writing data to and/or retrieving data from the component through a wireless data transmission protocol (for example, Bluetooth®, 802.11a, b, g, etc.). In another set of embodiments, the system includes a chip comprising a predetermined reaction site having a volume of less than about 2 ml or less than about 1 ml, where the predetermined reaction site is constructed and arranged to maintain at least one living cell at the site and a memory and/or data storage component capable of storing data, and where the system also includes an external reader/writer capable of writing data to and/or retrieving data from the component through radio waves. In yet another set of embodiments, the system includes a chip comprising a predetermined reaction site having a volume of less than about 2 ml or less than about 1 ml and a memory and/or data storage component capable of storing data, where the predetermined reaction site is constructed and arranged to maintain at least one living cell at the site, and where the system also includes an external reader/writer capable of writing data to and/or retrieving data from the component through optical interaction (for example, laser reading). In still another set of embodiments, the system includes a chip comprising a predetermined reaction site having a volume of less than about 2 ml or less than about 1 ml and a memory and/or data storage component capable of storing data, where the predetermined reaction site is constructed and arranged to maintain at least one living cell at the site, and where the system also includes an external reader/writer capable of writing data to and/or retrieving data from the component through a magnetic interaction.

In another aspect, the invention includes a method comprising the steps of providing a predetermined reaction site having a volume of less than about 2 ml or less than about 1 ml, where the predetermined reaction site constructed and arranged to maintain at least one living cell at the site, providing a material in the predetermined reaction site, collecting data related to and/or associated with the predetermined reaction site, and storing the data in a memory and/or data storage component associated with the chip. The method, in another set of embodiments, also includes a step of retrieving data stored in the component.

Other advantages and novel features of the invention will become apparent from the following detailed description of various non-limiting embodiments of the invention when considered in conjunction with the accompanying drawings. In cases where the present specification and a document incorporated by reference include conflicting disclosure, the present specification shall control. If two (or more) applications incorporated by reference include conflicting and/or inconsistent disclosure with respect to each other, then the later-filed application shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIG. 1 illustrates one embodiment of the invention, showing a memory component associated with a chip; and

FIG. 2 illustrates an interaction between an external reader/writer and a chip.

### DETAILED DESCRIPTION OF THE INVENTION

Each of the following commonly-owned applications directed to related subject matter and/or disclosing methods and/or devices and/or materials useful or potentially useful for the practice of the present invention is incorporated herein by reference: U.S. Provisional Patent Application Ser. No. 60/188,275, filed Mar. 10, 2000, entitled “Microreactor,” by Jury, et al.; U.S. patent application Ser. No. 09/707,852, filed Nov. 7, 2000, entitled “Microreactor,” by Jury, et al.; International Patent Application No. PCT/US01/07679, filed Mar. 9, 2001, entitled “Microreactor,” by Jury, et al., published as WO 01/68257 on Sep. 20, 2001; U.S. Provisional Patent Application Ser. No. 60/282,741, filed Apr. 10, 2001, entitled “Microfermentor Device and Cell Based Screening Method,” by Zarur, et al.; U.S. patent application Ser. No. 10/119,917, filed Apr. 10, 2002, entitled “Microfermentor Device and Cell Based Screening Method,” by Zarur, et al., published as 2003/0077817 on Apr. 24, 2003;-International Patent Application No. PCT/US02/11422, filed Apr. 10, 2002, entitled “Microfermentor Device and Cell Based Screening Method,” by Zarur, et al., published as WO 02/083852 on Oct. 24, 2002; U.S. Provisional Patent Application Ser. No. 60/386,323, filed Jun. 5, 2002, entitled “Materials and Reactors having Humidity and Gas Control,” by Rodgers, et al.; U.S. Provisional Patent Application Ser. No. 60/386,322, filed Jun. 5, 2002, entitled “Reactor Having Light-Interacting Component,” by Miller, et al.; U.S. patent application Ser. No. 10/223,562, filed Aug. 19, 2002, entitled “Fluidic Device and Cell-Based Screening Method,” by Schreyer, et al.; U.S. Provisional Patent Application Ser. No. 60/409,273, filed Sep. 9, 2002, entitled “Protein Production and Screening Methods,” by Zarur, et al.; U.S. patent application Ser. No. 10/457,048, filed Jun. 5, 2003, entitled “Reactor Systems Responsive to Internal Conditions,” by Miller, et al.; U.S. patent application Ser. No. 10/456,934, filed Jun. 5, 2003, entitled “Systems and Methods for Control of Reactor Environments,” by Miller, et al.; U.S. patent application Ser. No. 10/456,133, filed Jun. 5, 2003, entitled “Microreactor Systems and Methods,” by Rodgers, et al.; U.S. patent application Ser. No. 10/457,049, filed Jun. 5, 2003, entitled “Materials and Reactor Systems having Humidity and Gas Control,” by Rodgers, et al,. published as 2004/0058437 on Mar. 25, 2004; International Patent Application No. PCT/US03/17816, filed Jun. 5, 2003, entitled “Materials and Reactor Systems having Humidity and Gas Control,” by Rodgers, et al., published as WO 03/103813 on Dec. 18, 2003; U.S. patent application Ser. No. 10/457,015, filed Jun. 5, 2003, entitled “Reactor Systems Having a Light-Interacting Component,” by Miller, et al., published as 2004/0058407 on Mar. 25, 2004; International Patent Application No. PCT/US03/18240, filed Jun. 5, 2003, entitled “Reactor Systems Having a Light-Interacting Component,” by Miller, et al., published as WO 03/104384 on Dec. 18, 2003; U.S. patent application Ser. No. 10/457,017, filed Jun. 5, 2003, entitled “System and Method for Process Automation,” by Rodgers, et al.; U.S. patent application Ser. No. 10/456,929, filed Jun. 5, 2003, entitled “Apparatus and Method for Manipulating Substrates,” by Zarur, et al.; U.S. patent application Ser. No. 10/633,448, filed Aug. 1, 2003, entitled “Microreactor,” by Jury, et al; International Patent Application No. PCT/US03/25956, filed Aug. 19, 2003, entitled “Determination and/or Control of Reactor Environmental Conditions,” by Miller, et al., published as WO 2004/016727 on Feb. 26, 2004; U.S. patent application Ser. No. 10/664,046, filed Sep. 16, 2003, entitled “Determination and/or Control of Reactor Environmental Conditions,” by Miller, et al.; International Patent Application No. PCT/US03/25907, filed Aug. 19, 2003, entitled “Systems and Methods for Control of pH and Other Reactor Environmental Conditions,” by Miller, et al., published as WO 2004/016729 on Feb. 26, 2004; U.S. Patent Application Ser. No. 60/498,981, filed Aug. 29, 2003, entitled “Rotatable Reactor Systems and Methods,” by Zarur, et al.; U.S. Patent Application Ser. No. 60/499,124, filed Aug. 29, 20003, entitled “Reactor with Memory Component,” by Zarur, et al.; U.S. patent application Ser. No. 10/664,068, filed Sep. 16, 2003, entitled “Systems and Methods for Control of pH and Other Reactor Environmental Conditions,” by Miller, et al.; International Patent Application No. PCT/US03/25943, filed Aug. 19, 2003, entitled “Microreactor Architecture and Methods,” by Rodgers, et al.; a U.S. patent application filed on Sep. 16, 2003, entitled “Microreactor Architecture and Methods,” by Rodgers, et al.; a U.S. patent application filed on Jun. 7, 2004, entitled “Control of Reactor Environmental Conditions,” by Rodgers, et al.; a U.S. patent application filed on Jun. 7, 2004, entitled “System and Method for Process Automation,” by Rodgers, et al.; an International Patent Application filed on Jun. 7, 2004, entitled “System and Method for Process Automation,” by Rodgers, et al.; a U.S. patent application filed on Jun. 7, 2004, entitled “Apparatus and Method for Manipulating Substrates,” by Zarur, et al.; an International Patent Application filed on Jun. 7, 2004, entitled “Apparatus and Method for Manipulating Substrates,” by Zarur, et al.; an International Patent Application filed on Jun. 7, 2004, entitled “Reactor with Memory Component,” by Zarur, et al.; a U.S. patent application filed on Jun. 7, 2004, entitled “Gas Control in a Reactor,” by Rodgers, et al.; a U.S. Design patent application filed on Jun. 7, 2004, entitled “Reactor and Chip,” by Russo, et al.; a U.S. patent application filed on Jun. 7, 2004, entitled “Reactor Mixing” by Johnson, et al.; and a U.S. patent application filed on Jun. 7, 2004, entitled “Reactor Mixing Apparatus and Method,” by MacGregor.

The present invention provides techniques for conveniently and reliably storing and/or retrieving data associated with a chemical, biological, or biochemical chip or reactor. The data can pertain to the reactor; to chemical, biological, or biochemical species introduced into, taken from, or otherwise associated with the reactor; to conditions to which the reactor and/or some or all of its contents has been, is being, or will be exposed; or the like. In the invention, data can be stored to and/or retrieved from a data storage component associated with the chip itself. A data storage component is associated with a chip, in accordance with the invention, if the data storage component is fastened to, embedded within, or integral with the chip or otherwise will reliably travel with the chip as the chip is moved from place to place in the environment in which it is used, so that any data written to or retrieved from a particular data storage component will be reflective of some aspect or condition of the chip with which it is associated, and/or of one or more species with which the chip has been, is, or will be associated.

A variety of chips can benefit from data storage components of the invention. As an example, a suitable chip may contain a reaction site having a volume of less than about 2 ml or less than about 1 ml. In certain embodiments, the chip may be constructed in such a way as to be able to support a living cell. The chip may be used for imaging or analysis, or the chip may be used to facilitate a chemical or biological reaction, which may be light-sensitive or light-activated in certain cases. Other facilitated reactions may include the production and/or consumption of a chemical or biological species. In some embodiments, the chip may include more than one component or component type, and/or more than one reaction site.

A “chemical, biological, or biochemical reactor chip,” (also referred to, equivalently, simply as a “chip”) as used herein, is an integral article that includes one or more reactors. “Integral article” means a single piece of material, or assembly of components integrally connected with each other. As used herein, the term “integrally connected,” when referring to two or more objects, means objects that do not become separated from each other during the course of normal use, e.g., cannot be separated manually; separation requires at least the use of tools, and/or by causing damage to at least one of the components, for example, by breaking, peeling, etc. (separating components fastened together via adhesives, tools, etc.). A chip of the invention may have a maximum dimension of less than 50 cm or less than 20 cm, and may have a width to length (maximum dimension) ratio of between 1:5 and 1:1.2, with a thickness significantly less than the length or width, e.g. less than one-fifth of the width.

A chip can be connected to or inserted into a larger framework defining an overall reaction system, for example, a high-throughput system. The system can be defined primarily by other chips, chassis, cartridges, cassettes, and/or by a larger machine or set of conduits or channels, sources of reactants, cell types, and/or nutrients, inlets, outlets, sensors, actuators, and/or controllers. Typically, the chip can be a generally flat or planar article (i.e., having one dimension that is relatively small compared to the other dimensions); however, in some cases, the chip can be a non-planar article, for example, the chip may have a cubical shape, a curved surface, a solid or block shape, etc.

As used herein, a “membrane” is a thin sheet of material, typically having a shape such that one of the dimensions is substantially smaller than the other dimensions, that is permeable to at least one substance in an environment to which it is or can be exposed. In some cases, the membrane may be generally flexible or non-rigid. As an example, a membrane may be a rectangular or circular material with a length and width on the order of millimeters, centimeters, or more, and a thickness of less than a millimeter, and in some cases, less than 100 microns, less than 10 microns, or less than 1 micron or less. The membrane may define a portion of a reaction site and/or a reactor, or the membrane may be used to divide a reaction site into two or more portions, which may have volumes or dimensions which are substantially the same or different. Non-limiting examples of substances to which the membrane may be permeable to include water, O₂, CO₂, or the like. As an example, a membrane may have a permeability to water of less than about 1000 (g micrometer/m² day), 900 (g micrometer/m² day), 800 (g micrometer/m² day), 600 (g micrometer/m² day) or less; the actual permeability of water through the membrane may also be a function of the relative humidity in some cases.

Some membranes may be semipermeable membranes, which those of ordinary skill in the art will recognize to be membranes permeable with respect to at least one species, but not readily permeable with respect to at least one other species. For example, a semipermeable membrane may allow oxygen to permeate across it, but not allow water vapor to do so, or may allow water vapor to permeate across it, but at a rate that is at least an order of magnitude less than that for oxygen. Or a semipermeable membrane may be selected to allow water to permeate across it, but not certain ions. For example, the membrane may be permeable to cations and substantially impermeable to anions, or permeable to anions and substantially impermeable to cations (e.g., cation exchange membranes and anion exchange membranes). As another example, the membrane may be substantially impermeable to molecules having a molecular weight greater than about 1 kilodalton, 10 kilodaltons, or 100 kilodaltons or more. In one embodiment, the membrane may be impermeable to cells, but be chosen to be permeable to varied selected substances; for example, the membrane may be permeable to nutrients, proteins and other molecules produced by the cells, waste products, or the like. In other cases, the membrane may be gas impermeable. Some membranes may be transparent to particular light (e.g. infrared, UV, or visible light; light of a wavelength with which a device utilizing the membrane interacts; visible light if not otherwise indicted). Where a membrane is substantially transparent, it absorbs no more than 50% of light, or in other embodiments no more than 25% or 10% of light, as described more fully herein. In some cases, a membrane may be both semipermeable and substantially transparent. The membrane, in one embodiment, may be used to divide a reaction site constructed and arranged to support cell culture from a second portion, for example, a reservoir. For example, a reaction site may be divided into three portions, four portions, or five portions. For instance, a reaction site may be divided into a first cell culture portion and a second cell culture portion flanking a first reservoir portion and two additional reservoir portions, one of which is separated by a membrane from the first cell culture portion and the other of which is separated by a membrane from the second cell culture portion. Of course, those of ordinary skill in the art will be able to design other arrangements, having varying numbers of cell culture portions, reservoir portions, and the like, as described herein.

As used herein, a “reactor” is the combination of components including a reaction site, any chambers (including reaction chambers and ancillary chambers), channels, ports, inlets and/or outlets (i.e., leading to or from a reaction site), sensors, actuators, processors, controllers, membranes, and the like, which, together, operate to contain, promote and/or monitor a biological, chemical, and/or biochemical reaction, interaction, operation, or experiment at a reaction site, and which can be part of a chip. For example, a chip may include at least 5, at least 10, at least 20, at least 50, at least 100, at least 500, or at least 1,000 or more reactors. Examples of reactors include chemical or biological reactors and cell culturing devices, as well as the reactors described in International Patent Application No. PCT/US01/07679, filed Mar. 9, 2001, entitled “Microreactor,” by Jury, et al., published as WO01/68257 on Sep. 20, 2001, incorporated herein by reference. Reactors can include one or more reaction sites or compartments. The reactor may be used for any chemical, biochemical, and/or biological purpose, for example, cell growth, pharmaceutical production, chemical synthesis, hazardous chemical production, drug screening, materials screening, drug development, chemical remediation of warfare reagents, or the like. For example, the reactor may be used to facilitate very small scale culture of cells or tissues. In one set of embodiments, a reactor of the invention comprises a matrix or substrate of a few millimeters to centimeters in size, containing channels with dimensions on the order of, e.g., tens or hundreds of micrometers. Reagents of interest may be allowed to flow through these channels, for example to a reaction site, or between different reaction sites, and the reagents may be mixed or reacted in some fashion. The products of such reactions can be recovered, separated, and treated within the reactor or chip in certain cases.

As used herein, a “reaction site” is defined as a site within a reactor that is constructed and arranged to produce a physical, chemical, biochemical, and/or biological reaction during use of the reactor. More than one reaction site may be present within a reactor or a chip in some cases, for example, at least one reaction site, at least two reaction sites, at least three reaction sites, at least four reaction sites, at least 5 reaction sites, at least 7 reaction sites, at least 10 reaction sites, at least 15 reaction sites, at least 20 reaction sites, at least 30 reaction sites, at least 40 reaction sites, at least 50 reaction sites, at least 100 reaction sites, at least 500 reaction sites, or at least 1,000 reaction sites or more may be present within a reactor or a chip. The reaction site may be defined as a region where a reaction is allowed to occur; for example, a reactor may be constructed and arranged to cause a reaction within a channel, one or more compartments, at the intersection of two or more channels, etc. The reaction may be, for example, a mixing or a separation process, a reaction between two or more chemicals, a light-activated or a light-inhibited reaction, a biological process, and the like. In some embodiments, the reaction may involve an interaction with light that does not lead to a chemical change, for example, a photon of light may be absorbed by a substance associated with the reaction site and converted into heat energy or re-emitted as fluorescence. In certain embodiments, the reaction site may also include one or more cells and/or tissues. Thus, in some cases, the reaction site may be defined as a region surrounding a location where cells are to be placed within the reactor, for example, a cytophilic region within the reactor.

With respect to chips with data storage components, any type of data may be included in the data storage component, and the data may be added to the chip at any time, for example, before, during, or after one or more experiments have been performed on the chip. In some cases, data may be pre-recorded in the data storage component before an experiment is performed on the chip. In other cases, data may be written to the chip during and/or after an experiment is performed on the chip. In some cases, the data storage component may include both pre-recorded data, and data that is added during or after use of the chip.

As used herein, a “memory component” or a “data storage component” is defined as an element that can be reliably uniquely associated with a chip (or predetermined set of chips), constructed and arranged to allow data to be stored to and/or retrieved from the element.

A data storage compartment is associated with a chip, in accordance with one embodiment of the invention, if the data storage compartment is fastened to, embedded within, or integral with the chip, or otherwise will reliably travel with the chip as the chip is moved from place to place and the environment in which it is used, so that any data written to or retrieved from a particular data storage compartment will be reflective of some aspect or condition of the chip with which it is associated, and/or of one or more species with which the chip has been, is, or will be associated.

Various memory and/or data storage components are suitable and may include, but are not limited to, silicon integrated circuits, magnetic media, optical media, radio-frequency tags, smart cards, barcodes, and other kinds of data storage devices. In one embodiment, the data storage component is one to which data can be written and from which data can be read, repeatedly, such as a computer-readable medium, for example, a medium that stores information through electronic properties, magnetic properties, optical properties, etc. of the medium. Examples of computer-readable media include, but are not limited to, silicon and other semiconductor microchips or integrated circuits, bar codes, radio frequency tags or circuits, compact discs (e.g., in CD-R or CD-RW formats), digital versatile discs (e.g., in DVD+R, DVD−R, DVD+RW, or DVD−RW formats), insertable memory devices (e.g., memory cards, memory chips, memory sticks, memory plugs, etc.), “flash” memory, magnetic media (e.g., magnetic strips, magnetic tape, DATs, tape cartridges, etc.), floppy disks (e.g., 5.25 inch or 90 mm (3.5 inch) disks), optical disks, OCR readers, laser scanners, and the like. In one set of embodiments, the data storage component may be reversibly attached to and removed from the chip. In some embodiments, the data storage component may be volatile, i.e., some power is required by the data storage component to maintain the data therein. In other embodiments, however, the data storage component is non-volatile.

In one embodiment, the memory and/or data storage component includes a data storage chip. As used herein, a “data storage chip” is a microchip or microprocessor to which data can be stored and/or retrieved. Typically, the data storage chip comprises a semiconductor and often contains electronic circuitry. Examples of typical semiconductors for data storage chips include, but are not limited to, silicon, germanium, Group III-V compounds (e.g., GaAs, InAs, GaP, InP, GaN, etc.), Group II/VI, Group III/V and Group IV semiconductors such as CdS, CdSe, InP, GaAs, and the like. Other semiconductor materials are described below. It should be understood that, as used herein, a “data storage chip” is to be distinguished from a “chip,” as defined above (i.e., an integral article that includes one or more reactors). In particular, in one set of embodiments, a chip of the invention can include one or more data storage chips.

As used herein, a “processor” or a “microprocessor” is any component or device able to receive a signal from one or more sensors, store the signal, and/or convert the signal into one or more responses for one or more actuators, for example, by using a mathematical formula or an electronic or computational circuit. The signal may be any suitable signal indicative of the environmental factor determined by the sensor, for example a pneumatic signal, an electronic signal, an optical signal, a mechanical signal, etc. The processor may be any device suitable for determining a response to the signal, for example, a mechanical device or an electronic device such as a semiconductor chip. The processor may be embedded and integrally connected with the reaction site or chip or separate from the reaction site or chip, depending on the application. In one embodiment, the processor is programmed with a process control algorithm, which can, for example, take an incoming signal from a sensor and convert the signal into a suitable output for an actuator. Any suitable algorithm(s) may be used within the processor, for example, a PID control system, a feedback or feedforward system, a fuzzy logic control system etc. The processor may be programmed or otherwise designed to control an environmental condition within the reaction site, for example, by manipulation of an actuator.

As used herein, an “actuator” is a device able to affect the environment within or proximate to one or more reaction sites, or in an inlet or outlet in fluid communication with one or more reaction sites. The actuator may be separate from, or integrally connected to the reaction site or chip. For example, in some embodiments, the actuator may include a valve or a pump able to control, alter, and/or prevent the flow of a substance or agent into or out of the reaction site, for example, a chemical solution, a buffering solution, a gas such as CO₂ or O₂, a nutrient solution, a saline solution, an acid, a base, a solution containing a carbon source, a nitrogen source, an inhibitor, a promoter, a hormone, a growth factor, an inducer, etc. The substance to be transported will depend on the specific application. In some cases, the pump may be external of the chip. As one example, the actuator may selectively open a valve that allows CO₂ or O₂ to enter the reaction site. In other cases, the pump may be internal of the chip. For example, the pump may be a piezoelectric pump or a mechanically-activated pump, e.g., the pump may be activated by pressure, electrical stimulation, etc. In one embodiment, the pump is activated by producing a gas within the chip, which may cause fluid flow within the chip; as examples, the gas may be produced by directing light such as laser light at a reactant to start a gas-producing reaction, or the gas may be produced by applying an electric current to a reactant (for instance, an electric current may be applied to water to produce gas). As another example, the actuator may include a pumping system that can create a fluid connection with a reaction site as necessary.

More than one memory and/or data storage component may be present in association with a chip in some cases. For example, one or more memory and/or data storage components may be present within or on the chip, for instance, one or more memory and/or data storage components per reaction site. The data may be, for example, data related to a process or processes taking place within one or more of the reaction sites, identification data, parametric data (for example, data concerning when and where the chip was loaded, the contents of the chip, etc.) or the like. In some embodiments, the data may include physical, chemical, physicochemical, biological or biochemical data related to or associated with the reaction site. For example, the data may include experimental data, protocols and/or results, good manufacturing practices (“GMP”) data, data relevant to regulatory agencies, or the like.

In one aspect of the invention, data within the data storage component may be accessed externally from an external data interface. The external data interface may be positioned anywhere where it is able to establish communication with the data storage component of the chip. As used herein, an “external reader/writer” or an “external data interface” is a component or module capable of interfacing with a memory and/or data storage component of a chip to store and/or retrieve information or data. In one set of embodiments, the external data interface may be able to write data to the data storage component; in another set of embodiments, the external data interface may be able to read data to the storage component (“read only”); and in still another set of embodiments, the external data interface may be able to both read and write data to and from the data storage component. In some cases, data may be written to the data storage component multiple times (“rewritable”).

Data may be transmitted to/from the data storage component to the external data interface through any suitable method, for example, using any suitable data transfer protocol known to those of ordinary skill in the art. For example, data may be transferred by magnetic interactions, such as by using a magnetic medium; electrical interactions (i.e., through an electrical connection, for example, by inserting the chip and/or the data storage component into a socket on an external data interface); light interactions (including laser interactions), for example, an optical medium; radio frequency interaction, for example, a radio-frequency tag; etc. Examples of data transfer protocols include, but are not limited to, Bluetooth® data transfer protocols 802.11a, b, or g, etc., ftp protocols, Internet protocols and the like.

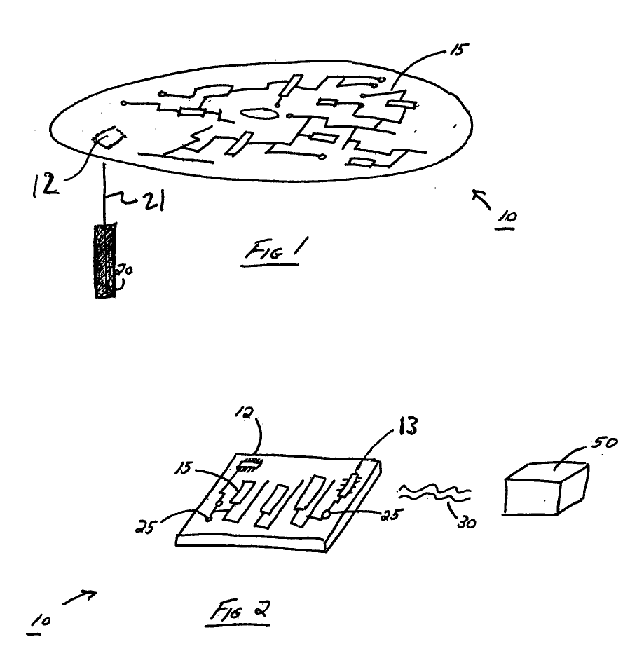

As one particular example of data transfer, as illustrated in FIG. 1, in a substantially planar chip 10 having a series of microfluidic elements 15 thereon (which may include one or more reaction sites, channels, ports, membranes, data storage components, or the like), data may be read from and/or written to the chip using a laser 20 producing a laser beam 21. In this embodiment, a data storage component 12 is on the underside of chip 10, and shown in broken line. The data written to the data storage compartment may be, for example, data representing reaction conditions or experimental protocols for chip 10, experimental or a parametric data, information about species introduced to or removed from the chip, etc. The data may be written to and/or read from, the underside of chip 10 using laser 20 during, before, or after an experiment is conducted on the chip. The chip, as illustrated in FIG. 1, may be fabricated such that it has the same general dimensions as a compact disc or a mini-compact disc, etc.

Another example of an embodiment of the invention is illustrated in FIG. 2. In this figure, chip 10 includes a plurality of microfluidic elements 15 and multiple data storage components 12 and 13. Chip 10 also includes connecting element 25 connecting one of the microfluidic elements 15 to a data storage component 12. Connecting element 25 may include, for example, a sensor, an actuator, a processor, or any combination of these (as further described below), and may be used to transfer data or other information between the microfluidic element and the data storage component. For example, if connecting element 25 is a sensor, then data from the sensor may be sent through connecting element 25 to data storage component 12 to be recorded. As another example, if connecting element 25 is an actuator, then a signal from data storage component 12 may be used by the actuator to alter an environmental factor within microfluidic element 15 (for example, within one or more reaction sites). In the particular embodiment shown in FIG. 2, chip 10 is fabricated such that it has the general dimensions of a microplate as is commonly used in the art. Also shown in FIG. 2 is data storage component 13, which can transmit data to and/or from an external data interface 50, e.g., using a wireless transmission protocol (represented by 30), which may be, for instance, radio waves, infrared waves, a magnetic field, optical waves or a laser, etc.

Many embodiments and arrangements of the disclosed devices are described with reference to a chip, or to a reactor, and those of ordinary skill in the art will recognize that the presently disclosed subject matter can apply to either or both. For example, a channel arrangement may be described in the context of one, but it will be recognized that the arrangement can apply in the context of the other (or, typically, both: a reactor which is part of a chip). It is to be understood that all descriptions herein that are given in the context of a reactor or chip apply to the other, unless inconsistent with the description of the arrangement in the context of the definitions of “chip” and “reactor” herein. It should also be understood that the chips and reactors disclosed herein may have a wide variety of different configurations. For example, a chip may be formed from a single material, or the chip may contain more than one type of reactor, reservoir and/or agent.

As used herein, a “channel” is a conduit associated with a reactor and/or a chip (within, leading to, or leading from a reaction site) that is able to transport one or more fluids specifically from one location to another, for example, from an inlet of the reactor or chip to a reaction site, e.g., as further described below. Materials (e.g., fluids, cells, particles, etc.) may flow through the channels, continuously, randomly, intermittently, etc. The channel may be a closed channel, or a channel that is open, for example, open to the external environment surrounding the reactor or chip containing the reactor. The channel can include characteristics that facilitate control over fluid transport, e.g., structural characteristics (e.g., an elongated indentation), physical/chemical characteristics (e.g., hydrophobicity vs. hydrophilicity) and/or other characteristics that can exert a force (e.g., a containing force) on a fluid when within the channel. The fluid within the channel may partially or completely fill the channel. In some cases the fluid may be held or confined within the channel or a portion of the channel in some fashion, for example, using surface tension (i.e., such that the fluid is held within the channel within a meniscus, such as a concave or convex meniscus). The channel may have any suitable cross-sectional shape that allows for fluid transport, for example, a square channel, a circular channel, a rounded channel, a rectangular channel (e.g., having any aspect ratio), a triangular channel, an irregular channel, etc. The channel may be of any size within the reactor or chip. For example, the channel may have a largest dimension perpendicular to a direction of fluid flow within the channel of less than about 1000 micrometers in some cases, less than about 500 micrometers in other cases, less than about 400 micrometers in other cases, less than about 300 micrometers in other cases, less than about 200 micrometers in still other cases, less than about 100 micrometers in still other cases, or less than about 50 or 25 micrometers in still other cases. In some embodiments, the dimensions of the channel may be chosen such that fluid is able to freely flow through the channel, for example, if the fluid contains cells. The dimensions of the channel may also be chosen in certain cases, for example, to allow a certain volumetric or linear flowrate of fluid within the channel. In one embodiment, the depth of other largest dimension perpendicular to a direction of fluid flow may be similar to that of a reaction site to which the channel is in fluid communication with. Of course, the number of channels, the shape or geometry of the channels, and the placement of channels within the chip can be determined by those of ordinary skill in the art.

In some embodiments, the reaction site may be defined by geometrical considerations. For example, the reaction site may be defined as a compartment in a reactor, a channel, an intersection of two or more channels, or other location defined in some fashion (e.g., formed or etched within a substrate that can define a reactor and/or chip). Other methods of defining a reaction site are also possible. In some embodiments, the reaction site may be artificially created, for example, by the intersection or union of two or more fluids (e.g., within one or several channels), or by constraining a fluid on a surface, for example, using bumps or ridges on the surface to constrain fluid flow. In other embodiments, the reaction site may be defined through electrical, magnetic, and/or optical systems. For example, a reaction site may be defined as the intersection between a beam of light and a fluid channel.

The volume of the reaction site or compartment can be very small in certain embodiments. Specifically, the reaction site may have a volume of less than one liter, less than about 100 ml, les than about 10 ml, less than about 5 ml, less than about 3 ml, less than about 2 ml, less than about 1 ml, less than about 500 microliters, less than about 300 microliters, less than about 200 microliters, less than about 100 microliters, less than about 50 microliters, less than about 30 microliters, less than about 20 microliters or less than about 10 microliters in various embodiments. The reaction site may also have a volume of less than about 5 microliters, or less than about 1 microliter in certain cases. The reaction site may have any convenient size and/or shape. In another set of embodiments, the reaction site may have a dimension that is 500 microns deep or less, 200 microns deep or less, or 100 microns deep or less.

In some cases, cells can be present at the reaction site. Sensor(s) associated with the chip or reactor, in certain cases, may be able to determine the number of cells, the density of cells, the status or health of the cells, the cell type(s), the physiology of the cells, etc. In certain cases, the reactor can also maintain or control one or more environmental factors associated with the reaction site, for example, in such a way as to support a chemical reaction or a living cell. In one set of embodiments, a sensor may be connected to an actuator and/or a microprocessor able to produce an appropriate change in an environmental factor within the reaction site. The actuator may be connected to an external pump, the actuator may cause the release of a substance from a reservoir, or the actuator may produce sonic or electromagnetic energy to heat the reaction site, or selectively kill a type of cell susceptible to that energy. The reactor can include one or more than one reaction site, and one or more than one sensor, actuator, processor, and/or control system associated with the reaction site(s). It is to be understood that any reaction site or a sensor technique disclosed herein can be provided in combination with any combination of other reaction sites and sensors.

The following features can be provided in conjunction with apparatus of the invention, alone or in combination, depending upon the use of the apparatus and the purpose for which it is employed, and these features are selectable by those of ordinary skill in the art with the benefit of the instant disclosure. Many of these features are described in greater detail in one or more of the above-referenced patents or patent applications.

Reactors or chips of the invention can be provided with channels (which can be microfluidic), inlets and outlets can be provided to introduce material into reaction compartments or sites and/or to remove material therefrom (e.g. gases such as CO₂, CO, oxygen, hydrogen, NO, NO₂, water vapor, nitrogen, ammonia, acetic acid, liquids such as water, saline, cells, cell culture medium, blood or other bodily fluids, antibodies, pH buffers, solvents, hormones, carbohydrates, nutrients, growth factors, therapeutic agents (or suspected therapeutic agents), antifoaming agents (e.g., to prevent production of foam and bubbles), proteins, antibodies, and the like. Parallelization can be achieved by forming an array of multiple reactors and/or reaction sites within a chip, or via a plurality of chips. For example, an array of at least about 10 chips, at least about 30, 50, 75, 100, 200, 500, 750, or at least about 1,000 chips or more) may be operated in parallel, for example, through the use of robotics. Additionally, any embodiment described herein can be used in conjunction with a collection chamber connectable ultimately to an outlet of one or more reactors and/or reaction sites of a chip. The collection chamber may have a volume of greater than 10 milliliters or 100 milliliters in some cases. The collection chamber, in other cases, may have a volume of greater than 100 liters or 500 liters, or greater than 1 liter, 2 liters, 5 liters, or 10 liters.

In one set of embodiments of the invention, the chip or other reactor system is rotatable, i.e., designed to be turned about an axis passing through the object. The object may be rotationally symmetric, or at least generally rotationally symmetric. A rotatable chip of the invention can have a generally circular shape in one embodiment. In some cases, the rotatable chip may have the same general dimensions as a commercially available compact disc (CD) or digital versatile disc (DVD) (e.g., about 12 cm in diameter), a mini-CD (e.g., about 8 cm in diameter), etc.

In some cases, chips of the invention can be constructed and arranged such that they are able to be stacked in a predetermined, pre-aligned relationship with other, similar chips, such that the chips are all oriented in a predetermined way (e.g., all oriented in the same way) when stacked together. When a chip of the invention is designed to be stacked with other, similar chips, it often can be constructed and arranged such that at least a portion of the chip, such as a reaction site, is in fluidic communication with one or more of the other chips and/or reaction sites within other chips. This arrangement can find use in parallelization of chips, as discussed herein.

In one set of embodiments, the chip is constructed and arranged such that the chip is able to be stably connected to a microplate, for example, as defined in the 2002 SPS/ANSI proposed standard (e.g., a microplate having dimensions of roughly 127.76±0.50 mm by 85.48±0.50 mm). In another set of embodiments, one or more reaction sites may be positioned in association with a chip such that, when the chip is stably connected to other chips, one or more reaction sites of the chip are positioned or aligned to be in chemical, biological, or biochemical communication with, or chemically, biologically, or biochemically connectable with one or more reaction sites of the other chip(s) and/or one or more wells of the microplate(s). Chips of the invention also may be constructed and arranged such that at least one reaction site and/or reactor of the chip is in fluid communication with, and/or chemically, biologically, or biochemically connectable to an apparatus constructed and arranged to address at least one well of a microplate. Chips of the invention can be substantially liquid-tight in one set of embodiments. As used herein, a “substantially liquid-tight chip” or a “substantially liquid-tight reactor” is a chip or reactor, respectively, that is constructed and arranged, such that, when the chip or reactor is filled with a liquid such as water, the liquid is able to enter or leave the chip or reactor solely through defined inlets and/or outlets of the chip or reactor, regardless of the orientation of the chip or reactor, when the chip is assembled for use.

Chips of the invention can be fabricated using any suitable manufacturing technique for producing a chip having one or more reactors, each having one or multiple reaction sites, and the chip can be constructed out of any material or combination of materials able to support a fluidic network necessary to supply and define at least one reaction site. For example, the chip may be fabricated by etching silicon or other substrates, for example, via standard lithographic techniques. The chip may also be fabricated by patterning multiple layers on a substrate, or by using various known rapid prototyping or masking techniques. Examples of materials that can be used to form chips include polymers, glasses, metals, ceramics, inorganic materials, and/or a combination of these. In some cases, the chip may be formed out of a material that can be etched to produce a reactor, reaction site and/or channel. In some embodiments, a chip of the invention may be formed from or include a polymer, such as, but not limited to, polyacrylate, polymethacrylate, polycarbonate, polystyrene, polyethylene, polypropylene, polyvinylchloride, polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The chip may also be formed from composite materials, for example, a composite of a polymer and a semiconductor material. In some embodiments, the chip, or at least a portion thereof, is rigid, such that the chip is sufficiently sturdy in order to be handled by commercially-available microplate-handling equipment, and/or such that the chip does not become deformed after routine use. Those of ordinary skill in the art are able to select materials or a combination of materials for chip construction that meet this specification, while meeting other specifications for use for which a particular chip is intended.

In certain embodiments, the chip may include a sterilizable material. For example, the chip may be sterilizable in some fashion to kill or otherwise deactivate biological cells (e.g., bacteria), viruses, etc. therein, before the chip is used or re-used. For instance, the chip may be sterilized with chemicals, radiated (for example, with ultraviolet light and/or ionizing radiation), heat-treated, or the like. Appropriate sterilization techniques and protocols are known to those of ordinary skill in the art. For example, in one embodiment, the chip is autoclavable, i.e., the chip is constructed and arranged out of materials able to withstand commonly-used autoclaving conditions (e.g., exposure to temperatures greater than about 100° C. or about 120° C., often at elevated pressures, such as pressures of at least one atmosphere), such that the chip, after sterilization, does not substantially deform or otherwise become unusable. Another example of a sterilization technique is exposure to ozone.

In some embodiments of the invention, the chip, or a portion thereof, such as a data storage component, may be moisture-resistant, i.e., the chip or component can be exposed to water without adversely affecting the chip or component. For example, the chip or component could be exposed to a liquid comprising water, a humidified atmosphere (e.g., within an incubator), ice (e.g., within a freezer), or steam (e.g., within an autoclave), without substantial damage or deformation (i.e., such that the chip or component can no longer function for its intended use).

The chip can also include a variety of components for sensing, actuation, or other activity. For example, the chip may include components such as a membrane, a lens, a light source, a waveguide, a circuit such as an integrated circuit, a reservoir (e.g., for a solution), a micromechanical or a MEMS (“microelectromechanical system”) component, a control system, or the like, for example, as further described below. In some embodiments, at least one, two, three or more components are integrally connected to the chip. In certain embodiments, all of the components are integrally connected to the chip.

In one set of embodiments, a chip of the invention may include a structure adapted to facilitate the reactions or interactions that are intended to take place therein (e.g., within a reaction site). For example, where a chip is intended to function as one or more bioreactors for cell culturing, the chip may include structure(s) able to improve or promote cell growth. For instance, in some cases, a surface of a reaction site may be a surface able to promote cell binding or adhesion, or the reactor and/or reaction site within the chip may include a structure that includes a cell adhesion layer, which may include any of a wide variety of hydrophilic, cytophilic, and/or biophilic materials. As examples, the surface may be ionized, or coated and/or micropatterned with any of a wide variety of hydrophilic, cytophilic, and/or biophilic materials, for example, materials having exposed carboxylic acid, alcohol, and/or amino groups. Examples of materials that may be suitable for a cell adhesion layer include, but are not limited to, polyfluoroorganic materials, polyester, PDMS, polycarbonate, polystyrene, and aluminum oxide. As another example, the structure may include a layer coated with a material that promotes cell adhesion, for example, an RGD peptide sequence, or the structure may be treated in such a way that it is able to promote cell adhesion, for example, the surface may be treated such that the surface becomes relatively more hydrophilic, cytophilic, and/or biophilic. In some embodiments, it may be desired to modify the surface of a cell adhesion layer, for instance with materials that promote cell adhesion, for example, by attachment, binding, soaking or other treatments. Example materials that promote cell adhesion include, but are not limited to, fibronectin, laminin, albumin or collagen. In other embodiments, for example, where certain types of bacteria or anchorage-independent cells are used, the surface may be formed out of a hydrophobic, cytophobic, and/or biophobic material, or the surface may be treated in some fashion to make it more hydrophobic, cytophobic, and/or biophobic, for example, by using aliphatic hydrocarbons and/or fluorocarbons. In some embodiments of the invention, a reactor and/or a reaction site within a chip may be constructed and arranged to maintain an environment that promotes the growth of living cells. In embodiments where one or more cells are used in the reaction site, the cells may be any cell or cell type. For example, the cell may be a bacterium or other single-cell organism, a plant cell, or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some cases, the cell may be a genetically engineered cell. In certain embodiments, the cell may be a Chinese hamster ovarian (“CHO”) cell or a 3T3 cell. In some embodiments, more than one cell type may be used simultaneously, for example, fibroblasts and hepatocytes. In certain embodiments, cell monolayers, tissue cultures or cellular constructs (e.g., cells located on a non-living scaffold), and the like may also be used in the reaction site. The precise environmental conditions necessary in the reaction site for a specific cell type or types may be determined by those of ordinary skill in the art.

In some embodiments, the chip is constructed and arranged such that cells within the chip can be maintained in a metabolically active state, for example, such that the cells are able to grow and divide. For instance, the chip may be constructed such that one or more additional surfaces can be added to the reaction site, for example, as in a series of plates, or the chip may be constructed such that the cells are able to divide while remaining attached to a substrate. In some cases, the chip may be constructed such that cells may be harvested or removed from the chip, for example, through an outlet of the chip, or by removal of a surface from the reaction site, optionally without substantially disturbing other cells present within the chip. The chip may be able to maintain the cells in a metabolically active state for any suitable length of time, for example, 1 day, 1 week, 30 days, 60 days, 90 days, 1 year, or indefinitely in some cases.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

In the claims, as well as in the specification above, all transitional phrases such as “comprising,” “including carrying,” “having,” “containing,” “involving,” “holding,” and the like are to be understood to be open-ended, i.e., to mean including but not limited to. Only the transitional phrases “consisting of” and ” consisting essentially of” shall be closed or semi-closed transitional phrases, respectively, as set forth in the United States Patent Office Manual of Patent Examining Procedures, Section 2111.03.

1. An apparatus, comprising:
a chemical, biological, or biochemical reactor chip comprising a predetermined reaction site having a volume of less than about 2 ml; and
at least one data storage component associated with the chip.

2. The apparatus of claim 1, wherein the predetermined reaction site is constructed and arranged to maintain at least one living cell at the site.

3. The apparatus of claim 1, comprising a plurality of reaction sites.

4. The apparatus of claim 1, wherein the predetermined reaction site has a volume of less than about 500 microliters.

5. The apparatus of claim 1, wherein the predetermined reaction site has a volume of less than about 100 microliters.

6. The apparatus of claim 1, wherein the predetermined reaction site has a volume of less than about 10 microliters.

7. The apparatus of claim 1, wherein the predetermined reaction site has a volume of less than about 1 microliter.

8. The apparatus of claim 1, wherein the predetermined reaction site has a maximum dimension of less than about 1 cm.

9. The apparatus of claim 1, wherein the predetermined reaction site has a maximum dimension of less than about 1 mm.

10. The apparatus of claim 1, wherein the predetermined reaction site has a maximum dimension of less than about 100 micrometers.

11. The apparatus of claim 1, wherein the predetermined reaction site has a maximum dimension of less than about 10 micrometers.

12. The apparatus of claim 1, wherein the at least one data storage component comprises a computer-readable medium.

13. The apparatus of claim 1, wherein the at least one data storage component is non-volatile.

14. The apparatus of claim 1, wherein the at least one data storage component is volatile.

15. The apparatus of claim 1, wherein the at least one data storage component comprises a magnetic medium.

16. The apparatus of claim 15, wherein the magnetic medium comprises a magnetic strip.

17. The apparatus of claim 1, wherein the at least one data storage component comprises an optical medium.

18. The apparatus of claim 1, wherein the optical medium is laser-readable.

19. The apparatus of claim 1, wherein the at least one data storage component comprises a semiconductor chip.

20. The apparatus of claim 1, wherein the at least one data storage component comprises a silicon chip.

21. The apparatus of claim 1, wherein the at least one data storage component comprises a radio frequency tag.

22. The apparatus of claim 1, wherein the at least one data storage component comprises a bar code.

23. The apparatus of claim 1, wherein the at least one data storage component is reversibly attachable.

24. The apparatus of claim 1, wherein the at least one data storage component is rotatable.

25. The apparatus of claim 1, wherein the at least one data storage component is rewritable.

26. The apparatus of claim 1, wherein the at least one data storage component is read-only.

27. The apparatus of claim 1, wherein the predetermined reaction site comprises at least one substantially hydrophobic surface.

28. The apparatus of claim 1, wherein the predetermined reaction site comprises at least one substantially hydrophilic surface.

29. The apparatus of claim 1, wherein the predetermined reaction site comprises at least one substantially cytophobic surface.

30. The apparatus of claim 1, wherein the predetermined reaction site comprises at least one substantially cytophilic surface.

31. The apparatus of claim 1, wherein the at least one data component is able to store data associated with the reaction site.

32. The apparatus of claim 1, wherein the at least one data storage component comprises a compact disc or a digital versatile disc.

33. The apparatus of claim 1, wherein the at least one data storage component comprises flash memory.

34. The apparatus of claim 1, wherein the at least one data storage component comprises an integrated circuit.

35. The apparatus of claim 1, wherein the at least one data storage component is a memory stick.

36. The apparatus of claim 1, wherein the at least one data storage component is reversibly removable from the chip.

37. The apparatus of claim 1, wherein the at least one data storage component can be exposed to gamma rays -without deformation.

38. The apparatus of claim 1, wherein the at least one data storage component can be exposed to ultraviolet radiation without deformation.

39. The apparatus of claim 1, wherein the at least one data storage component can be heated to a temperature of at least about 37° C. without deformation.

40. The apparatus of claim 39, wherein the at least one data storage component can be heated to a temperature of at least about 100° C. without deformation.

41. The apparatus of claim 1, wherein the at least one data storage component is moisture-resistant.

42. A system, comprising the apparatus of claim 1, and an external data interface able to interface with the at least one data storage compartment.

43. A method, comprising:
transferring data to or from a chemical, biological, or biochemical reactor chip comprising a data storage component and a predetermined reaction site having a volume of less than about 2 ml.

44. The method of claim 43, wherein the data is associated with the reaction site.

45. The method of claim 44, wherein the data identifies the chip.

46. The method of claim 44, wherein the data comprises a measurement of an environmental condition of the reaction site.

47. The method of claim 43, wherein the data is transferred to an external data interface.

48. The method of claim 47, wherein the data is transferred to the external data interface through an electrical connection.

49. The method of claim 47, wherein the data is transferred to the external data interface through a wireless connection.

50. The method of claim 49, wherein the wireless connection is a Bluetooth transmission protocol.

51. The method of claim 47, wherein the data is transferred to the external data interface using a radio wave.

52. The method of claim 47, wherein the data is transferred to the external data interface using a light interaction.

53. The method of claim 47, wherein the data is transferred to the external data interface using a magnetic interaction.

54. A system, comprising:
a chemical, biological, or biochemical reactor chip comprising a predetermined reaction site and a data storage component; and
an external data interface able to interface with the data storage component.

55. The system of claim 54, wherein the external data interface is able to write data to the data storage component.

56. The system of claim 54, wherein the external data interface is able to read data from the data storage component.

57. A system, comprising:
a holder able to secure a chemical, biological, or biochemical reactor chip; and
an external data interface positioned to interface with a chip secured by the holder.

58. The system of claim 57, wherein the external data interface is positioned to magnetically interface with the chip.

59. The system of claim 57, wherein the external data interface is positioned to establish an electrical connection with the chip.

60. The system of claim 57, wherein the external data interface is positioned to interface with the chip through a wireless connection.

61. The system of claim 60, wherein the external data interface is positioned to interface with the chip through a radio wave.

62. The system of claim 60, wherein the external data interface is positioned to interface with the chip through a light interaction.

63. The system of claim 60, wherein the external data interface is positioned to interface with the chip through a laser interaction.

64. A method, comprising:
storing data to and/or retrieving data from a data storage component of a chemical, biological, or biochemical reactor chip comprising a predetermined reaction site having a volume of less than about 2 ml.

65. A method as in claim 64, comprising storing data to the data storage component.

66. A method as in claim 64, comprising retrieving data from the data storage component.

67. A method as in claim 64, comprising storing data to and retrieving data from the data storage component.
